(19) United States
(12) Patent Application Publication
(10) Pub. No.: US 2010/0093599 A1
(43) Pub. Date: Apr. 15, 2010

(54) SHINE CONTROL CLEANSER

(76) Inventors: JEFFREY WU, Princeton, NJ (US); Xufeng Wu, Shanghai (CN)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/249,368

(22) Filed: Oct. 10, 2008

(51) Int. Cl. A61K 8/64; A61K 8/36; A61Q 19/10; A61Q 19/00

(52) U.S. Cl. 514/2; 510/130

(57) ABSTRACT

This invention relates to shine control cleansing compositions, which are capable of controlling the appearance of oily and shiny skin for an extended period of time.

### FIELD OF THE INVENTION

This invention relates to a novel composition useful for the treatment of skin, and more particularly, to a composition for cleansing and providing shine control and oil control of oily mammalian skin.

### BACKGROUND OF THE INVENTION

Skin care products for problem skin are well known. Oily skin in particular, is shiny, thick and dull colored. Often chronically oily skin has coarse or enlarged pores, pimples and other embarrassing blemishes. Oily skin is also prone to blackheads. In this type of skin, the oil producing sebaceous glands are overactive and produce more oil than is needed. The oil flows out of the follicles and gives the skin an undesired greasy shine and feel. The pores are enlarged and the skin has a coarse look. Oily skin is common in teenagers, but it can occur at any age.

It is advantageous, therefore, to provide means for controlling the distribution of oil over the surface of human skin, with particular regard to skin characterized by an excessive secretion or presence of oil upon the surface and to affected skin areas of, for example, acne patients. It is also advantageous to provide a facial care cleansing composition which will assist the facial stratum corneum in maintaining barrier and water retention functions in spite of exposure to external factors such as washing, work, and recreation.

Existing rinse-off cleansing treatments and preparations tend to dry the skin and cause irritation. They can only provide a very transient relief to the oily or shiny skin via cleansing action or temporary physical oil absorption. Oil and shine will return quickly within a very short time such as one to two hours.

Therefore, there exists a need for a novel cleansing composition for controlling the shiny appearance of oily skin for an extended period of time.

### SUMMARY OF THE INVENTION

We have discovered that, unexpectedly, skin care cleansing compositions of a certain composition have the capability of controlling sebum flow, controlling or inhibiting the oily, shiny appearance of the skin and inhibiting the consequential disorders resulting therefrom, such as, for example, acne.

Preferably, the skin care cleansing compositions of this invention contain at least one hydrolyzed protein and a cleansing agent. Preferably, but optionally, the skin care cleansing compositions of this invention further contain a keratolytic agent and a thickening agent. The skin care cleansing compositions of this invention control the appearance of oil on the skin for an extended duration, e.g., at least 6 hours.

In another embodiment, the skin care cleansing compositions of this invention contain from about 0.05% to about 95.0% by weight of the composition of at least one hydrolyzed protein and from about 1.0 to about 90% by weight of a cleansing agent. Optionally, they may contain from about 0.05 to about 5% by weight of a thickening agent. The thickening agent is capable of imparting the appropriate viscosity to the cleanser compositions suitable for use in this invention.

This invention also provides a method of reducing the appearance of oil on the skin for at least 6 hours, which comprises topically applying to said skin a composition comprising at least one hydrolyzed protein and a cleansing agent.

### DETAILED DESCRIPTION OF THE INVENTION

As used herein, “topically applying” means directly laying on or spreading on outer skin, the scalp, or hair of a human or mammal, e.g., by use of the hands or an applicator such as a wipe, roller, or spray.

As used herein, “cosmetically acceptable” means that the ingredients the term describes are suitable for use in contact with tissues (e.g., the skin or hair) without undue toxicity, incompatibility, instability, irritation, allergic response, or the like.

As used herein, an “effective amount” means an amount of ingredient or composition sufficient to provide a desired benefit or cosmetic or therapeutic effect. The effective amount will vary with the area being treated; the age and skin or hair type of the user, the duration and nature of the treatment, the specific ingredients employed, and like factors.

In one embodiment, the compositions of this invention contain at least one hydrolyzed protein, a cleansing agent and optionally a thickener.

The hydrolyzed protein may be a fully hydrolyzed protein or a partially hydrolyzed protein. The hydrolyzed protein may contain rice protein or a protein derived from cereal grains such as but not limited to wheat, barley, maize, millets, sorghum, oats, rye, fonio, quinoa, buckwheat or a combination thereof. The hydrolyzed protein may make up from about 0.05 to about 95% by weight of the composition. More preferably the hydrolyzed protein contains a combination of hydrolyzed protein portion and non-hydrolyzed protein portion. In this embodiment, the hydrolyzed protein portion comprises about 0.05% to 95% of the total hydrolyzed protein. That is, the hydrolyzed protein comprises from about 0.05% to 100% by weight of total protein.

Another embodiment of the present invention is directed to a method for depositing a thin coating of rice protein on skin, comprising topically applying an effective amount of a delivery system composition comprising said rice protein and a deposition enhancer system comprising at least one cationic compound such as xanthan, chitosan, carboxymethyl guar, alginates, hydroxypropyl guar, carboxymethyl guar hydroxypropyltrimethylammonium chloride, guar hydroxypropyltrimethylammonium chloride, hydroxypropyl guar hydroxypropyltrimethylammonium chloride, or quaternium polymers or salts such as polyquaternium-39, polyquaternium-7 or combinations thereof.

Another embodiment of the present invention is directed to a method for depositing a thin coating of rice protein on skin, comprising topically applying an effective amount of a delivery system composition comprising said rice protein and a deposition enhancer system comprising at least one cationic compound and at least one anionic compound. The cationic compound can be selected from but is not limited to guar hydroxypropyltrimonium chloride, acrylaminopropyltrimonium chloride/acrylamide copolymer, quaternium polymers or salts such as polyquaternium-39, polyquaternium-7, and combinations thereof. The anionic compound may be selected from, but is not limited to, the group consisting of acrylate polymers, such as Carbopol Aqua SF1, and mixtures thereof.

Examples of cleansing agents include but are not limited to fatty acids such as lauric acid, myristic acid, palmitic acid, steraric acid, coconut oil with alkali, their salts or mixtures thereof, anionic surfactants such as sodium lauryl sulfate and sodium lauryl sulfonate, sugar sulfonate, nonionic surfactants, cationic surfactants, amypholytic surfactants, zwitterionic surfactants, and/or semi-polar surfactants, as is well known in the cleansing art. The surfactant can be insoluble (or soluble) and is present in the composition in the amount of from about 1.0 to 90% by weight of the composition.

Zwitterionic surfactants include derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic water-solubilizing group, e.g., carboxy, sulfonate, sulfate, phosphate, or phosphonate.

Cationic surfactants, for example, can contain amino or quaternary ammonium hydrophilic moieties that are positively charged when dissolved in an aqueous composition.

Examples of amphoteric surfactants include derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Anionic surfactants include but are not limited to alkyl and alkyl ether sulfates, sulfonates, sulfosuccinates, sacosinates, carboxylates, and isethionates.

Nonionic surfactants can be selected from compounds containing a hydrophobic moiety and a nonionic hydrophilic moiety. Examples of the hydrophobic moiety may include alkyl, alkyl aromatic, dialkyl siloxane, polyoxyalkylene, and fluoro-substituted alkyls. Examples of hydrophilic moieties may include polyoxyalkylenes, phosphine oxides, sulfoxides, amine oxides, and amides. Other examples of nonionic surfactants include alkyl polysaccharides such as alkyl polysaccharides.

In one embodiment, the composition contains a thickening agent. Examples of thickening agents include but are not limited to inorganic salts such as NaCl, NH₄Cl, KCl, Na₂SO₄, clays, silicas, magnesium aluminum silicate, cellulosic polymers such as hydroxyethylcellulose, and xanthan gum, fatty alcohols such as cetyl alcohol, fatty acid esters, fatty acid amides, ethers of polyethylene glycol or sorbitol polyethylene glycol, cocamide of mono- or di-ethanolamide, cocamidopropyl betaine, quaternium-46, hydroxyethyl cellulose, cocodimonium chloride, polyquaternium-6, polyquaternium-7, quaternium-18, PEG-18 glycerol oleate/cocoate, a mixture of acrylates/steareth-50 acrylate copolymer, laureth-3 propylene glycol, and combinations thereof.

In one embodiment of the present invention wherein a polymeric emulsifier such as, for example, polyethylene glycol-30 dipolyhydroxystearate (hereinafter “PEG 30”) or dimethicone copolyol, are used and water is used as the vehicle, an oil-in-water emulsion may be produced.

A keratolytic agent may be used in the composition in order to treat skin conditions such as acne, blackheads and other skin diseases or conditions such as dry skin or oily skin, as known in the skin care art. One or more known keratolytic agents such as retinoids (e.g., tretinoin, retinol and retinal), carboxylic acids including α-hydroxy acids (e.g., lactic acid, glycolic acid), β-hydroxy acids (e.g., salicylic acid), α-keto acids, acetic acid and trichloroacetic acid, 1-pyrrolidone-5-carboxylic acid, capryloyl salicylic acid, α-hydroxy decanoic acid, α-hydroxy octanoic acid, gluconolactone, methoxypropyl gluconamide, oxalic acid, malic acid, tartaric acid, mandelic acid, benzylic acid, gluconic acid, benzoyl peroxide, resorcinol, colloidal sulphur, selenium disulphide, sulfur, and phenol or a combination thereof may be used. From about 0.01% to about 10% by weight of a keratolytic agent may be used.

The composition may also include anti-inflammatory or soothing agents. Examples include steroidal anti-inflammatory agents such as but are not limited to corticosteroids such as hydrocortisone, hydrocortisone acetate, hydrocortisone butyrate, and others. Nonsteroidal anti-inflammatory agents may include but are not limited to ibuprofen, naproxen, ketprofen; and botanical extracts such as allantoin, aloe vera, alnus, alpha-bisabolol, arnica, artemisia capillaris, asiasarum root, birch, bisabolol, boswellia, calendula, matricaria (chamomilla recutita) extract, cnidium, comfrey, dipotassium, glycyrrhizinate, feverfew, fennel, galla rhois, green tea (camellia sinesis) extract, ginger root extract, hawthorn, houttuynia, hypericum, jujube, kiwi, licorice, magnolia, sapindus mukurossi fruit extract, olive, panthenol, panthenoic acid and its derivatives, dexpanthenol and ethyl panthenol, flavonoids such as unsubstituted flavanones, mono-substituted flavanones, and mixtures; chalcones selected from unsubstituted chalcones, mono-substituted chalcones, di-substituted chalcones, tri-substituted chalcones; isoflavones; coumarins; chromones; dicoumarols; chromanones; chromanols; and turmeric (curcuma longa) extract peppermint, philodendron, salvia, sasa albo-marginata, tocopheryl acetate, tocopheryl nicotinate, imidazoles, ketoconazole and elubiol, and those described in Gollnick, H. et al. 196(1) Dermatology Sebaceous Glands, Acne and Related Disorders, 119-157 (1998), which is incorporated by reference herein, and combinations, derivatives, isomers, or mixtures thereof. From about 0.005% to about 10% by weight of an anti-inflammatory agent may be used, preferably from about 0.05% to about 5% and most preferably from about 0.1% to about 2%.

The composition of this invention may also include anti-acne or anti-razor bumps actives. Examples of suitable anti-acne agents include, but are not limited to topical retinoids (tretinoin, isotretinoin, motretinide, adapalene, tazarotene, azelaic acid, retinol); salicylic acid; benzoyl peroxide; resorcinol; antibiotics such as tetracycline and isomers thereof, erythromycin, and mixtures thereof. Suitable amounts of anti-acne agents include, based upon the total weight of the composition, from about 0.01 percent to about 10 percent.

The composition may also include abrasives or skin exfoliators such as but not limited to polyethylene, silica, clays, acrylate polymers and metallic or ceramic particles, natural exfoliating ingredients such as corn cob, tamarind, crushed apricot kernel, sea salts with different shapes and sizes, e.g., such as less than 1000 microns, preferably less than 500 microns but larger than 20 microns, or a combination thereof may be used.

In another embodiment, the composition includes antipruritics and skin protectants, such as oatmeal, betaglucan, feverfew, soy and derivatives thereof, bicarbonate of soda, colloidal oatmeal, surfactant based colloidal oatmeal cleanser, Anagallis Arvensis, Oenothera Biennis, Verbena Officinalis, and the like. These antipruritics may be used in an amount, based upon the total weight of the cleansing composition, from about 0.01 percent to about 40 percent, and preferably from about 1 percent to about 5 percent.

A tanning agent may be used in the composition such as but not limited to dihydroxyacetone (DHA) or 1,3-dihydroxy-2-propanone and their esters or derivatives, and their mixtures thereof.

Another embodiment is directed to a method for de-pigmenting skin or evening skin tone comprising topically applying to skin having abnormal pigmentation a composition of the invention. The composition may also include skin bleaching and/or lightening agents such as hydroquinone, kojic acid, arbutin, ascorbic acid and derivatives, e.g., magnesium ascorbyl phosphate, sodium ascorbyl phosphate, ascorbyl glucoside, and ascorbyl glucosamine, and natural extracts such as mulberry extract, soybean trypsin inhibitors, soy, isoflavones, flavonoids, mushroom extracts, yeast and its derivatives and soy placental extracts, and enzymes with depigmentation benefits such as lignin peroxidase, papain, papaya, and their derivatives, and mixtures thereof.

The composition may also include anti-cellulite agents. Examples of anti-cellulite agents include but are not limited to, xanthine compounds such as caffeine, theophylline, theobromine, aminophylline and combinations thereof.

The composition may also include anti-wrinkle or anti-atrophy agents. Examples include sulfur containing D and L amino acids and their derivatives and salts, particularly the N-acetyl derivatives, N-acetyl-L-cysteine; thiols, such as ethane thiol; alpha-hydroxy acids (AHA) or beta-hydroxy acids such as salicylic acid and salicylic acid derivatives, lysophosphatidic acid, phytic acid, lipoic acid and phenol.

Compositions of the present invention may also include ingredients such as antimicrobial agents, such as quinolone, beta-lactam derived compounds, metronidazole, doxycycline, miconazole, clindamycin, erythromycin, zinc erythromycin, minocycline, tetracycline hydrochloride, gentamicin, ethambutol, pentamidine, kanamycin, lineomycin, methacycline, methenamine, neomycin, ketaconazole, octopirox, nystatin, tolnaftate, clotrimazole, phenoxyethanol, hexamidine, isethionate, chlorhexidine, zinc pyrithione, benzakonium chloride, benzathonium chloride, and their derivatives or combinations.

Compositions of the present invention may also include other ingredients such as but not limited to skin appearance modifying ingredients that help mask imperfections such as fine lines, wrinkles, dark marks of age spot, blemishes, pimples etc., including but not limited to light reflectants, refractants, or absorbers such as titanium dioxide, zinc oxide, alumina, calcium silicate, glycol dioleate, glycol distearate, sodium magnesium fluorosilicate, and iron oxides, silica, silicone, silicone derivatives, soft focusing light colored pigments such as mica, organic particulates, crystal lipids and colorants, and mixtures thereof.

Water, which makes up the remaining portion of the compositions of this invention, provides up to about 99.5% by weight of the compositions.

Other ingredients which may be included in the compositions of this invention include anti-oxidants or radical scavengers, preferred concentrations of which range from about 0.1% to about 10%, more preferably from about 1% to about 5% wt/wt of the composition. Examples of antioxidants for use herein include but not limited to tocopherol, tocopherol acetate, other esters of tocoperol, ascorbic acid and its salts, ascorbyl esters of fatty acids, ascorbic acid derivatives (e.g., magnesium ascorbyl phosphate, sodium ascorbyl phosphate, ascorbic glucoside, ascorbyl sorbate), butylated hydroxy benzoic acids and their salts, O-hydroxy-tetramethylchroman-carboxylic acid.

Yet other embodiments of the compositions include skin humectants or emollient agents. The humectant is preferably present in an amount of from about 0 percent to about 10 percent, more preferably from about 0.5 percent to about 5 percent, and most preferably from about 0.5 percent to about 3 percent, based on the overall weight of the composition. Examples of suitable humectants nonexclusively include: 1) water soluble liquid polyols selected from the group comprising glycerine, propylene glycol, hexylene glycol, butylene glycol, pentylene glycol, dipropylene glycol, and mixtures thereof; 2) polyalkylene glycol of the formula HO—(R″O)_{b}—H wherein R″ is an alkylene group having from about 2 to about 4 carbon atoms and b is an integer of from about 1 to about 10, such as PEG 4; 3) polyethylene glycol ether of methyl glucose of formula CH₃—C₆H₁₀O₅—(OCH₂ CH₂)_{c}—OH wherein c is an integer from about 5 to about 25; 4) urea; 5) fructose; 6) glucose; 7) honey; 8) lactic acid; 9) maltose; 10) sodium glucuronate; and 11) mixtures thereof, with glycerine being the preferred humectant.

Emollients may include but not limited to petrolatum, fatty acid lactates, and other hydroxyl acids, their salts and their esters, phospholipids such as Cocamidopropyl PG-Dimonium Chloride Phosphate.

Compositions of the invention may also include skin softening and firming agents such as DMAE and tetrahydroxypropyl ethylenediamine (e.g., Neutrol from BASF), or those described in, e.g., US Patent Publication No. US20060193815 A1.

The composition can also include the pore refining and minimizing agents, such as astringents, and collagen and elastin enhancers, such as witch hazel extract.

The composition can include external analgesics and local anesthetics, which nonexclusively include benzocaine, dibucaine, benzyl alcohol, camphor, capsaicin, capsicum, capsicum oleoresin, juniper tar, menthol, methyl nicotinate, methyl salicylate, phenol, resorcinol, turpentine oil, and mixtures thereof.

The composition can include antiperspirants and deodorants nonexclusively including aluminium chlorohydrates, aluminium zirconium chlorohydrates, and mixtures thereof.

The composition can include counterirritants nonexclusively including camphor, menthol, methyl salicylate, peppermint and clove oils, lemmonichtammol, and mixtures thereof.

The composition may also include those therapeutic components that are effective in the treatment of dandruff, seborrheic dermatitis, and psoriasis as well as the symptoms associated therewith. Examples of such suitable benefits agents nonexclusively include zinc pyrithione, anthralin, shale oil and derivatives thereof such as sulfonated shale oil, selenium sulfide, sulfur; salicylic acid; coal tar; povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazole nitrate and any possible stereo isomers and derivatives thereof, piroctone olamine (Octopirox); selenium sulfide; ciclopirox olamine; anti-psoriasis agents such as vitamin D analogs, e.g. calcipotriol, calcitriol, and tacaleitrol; vitamin A analogs such as esters of vitamin A, e.g. vitamin A palmitate, retinoids, retinols, and retinoic acid; corticosteroids such as hydrocortisone, clobetasone, butyrate, clobetasol propionate, or natural ingredients such as green tea, pearl powder, orchid, lemon, lotus leaf, mung bean, sage and ginkgo biloba, carbon, sea salt, sea or mineral mud, and mixtures thereof.

Compositions of the present invention may contain at least one pigment. A “pigment” is a compound(s) that can be taken up by epidermal cells, resulting in visually darker look to the skin or hair. Examples of such pigments include, but are not limited to, melanin and melanin derivatives (e.g, both melanin polymers and lower molecular weight water-soluble melanin derivatives); extracts from natural sources containing pigments (e.g., brown pigments from plants from the Hedychium genus or Bearberry genus or yellow, orange and red pigments, from plants containing carotenoids or canthaxanthins); or synthetic chemicals such as compounds containing copper (e.g., copper salts such as CuCl₂) or synthetic carotenoids or canthaxantins. Examples of synthetic melanin derivatives are disclosed in U.S. Pat. Nos. 5,618,519, 5,384,116, and 5,227,459. Examples of soluble melanin derivatives are disclosed in U.S. Pat. Nos. 5,744,125, 5,225,435, 5,218,079, and 5,216,116. Examples of commercially available soluble melanin derivatives include Melasyn-100® from Sanmar laboratories, Inc. (Elmsford, N.Y.) and MelanZe® from Zylepsis (Ashford, Kent, United Kingdom).

The amount of pigment(s) present in the composition will depend on the type of pigment(s) used. The pigments typically will be present in the composition in an amount from about 0.001% to about 20% by weight, in particular in an amount from about 0.005% to about 5% by weight.

Compositions of the present invention may contain depigmentation agents. Depigmentation agents include, but are not limited to: soybean trypsin inhibitors or natural extract containing a soybean trypsin Inhibitor, Bowman-Birk Inhibitor or a natural extract containing a Bowman-Birk Inhibitor, soy extract, soy isoflavones, isoflavonoids and their derivatives; retinoids such as retinol, retinoic acid and their derivatives; kojic acid; kojic dipalmitate; hydroquinone; arbutin; transexamic acid; vitamins such as but not limited to vitamin B complex, including thiamine, nicotinic acid, niacin, biotin, pantothenic acid, choline, riboflavin, vitamin B3, B6, B12, pyridoxine, inositol, carnitine; vitamins A, C, D, E, K and their derivatives such as vitamin A palmitate and pro-vitamins, e.g. (i.e. panthenol (pro vitamin B5) and panthenol triacetate), its salts & derivatives and mixtures vitamin, Vitamin K, azelaic acid, ascorbic acid polypeptide, pearls; linolenic acid, glycyrrhetinic acid, and linoleic acid; placertia; licorice; and extracts such as but not limited to chamomile, Osage orange (Mac lura pomifera) heartwood, bearberry (arctostaphylos uva-ursi), mulberry, apple phenon, lemon (citrus medica limonum) peel, grape seed, and green tea; and salts, esters, and other derivatives thereof. Other depigmenting ingredients can include Dragostat 11, papain, mushroom extract, Melanostat, Yokinoshita, Symwhite 377, dioic acid, peptide manganese complex or tyrosinase inhibiting enzymatic proteases. Perilla extract is disclosed as a whitening agent in U.S. Pat. No. 5,980,904 and Japanese Publication Nos. 07025742, 07187989, 10265322, 2001163759, and 2001181173. Coconut fruit extract is disclosed as a whitening agent in Japanese Patent No. 2896815B2. An extract of the spongy mass of coconut tissue is employed in a tanning sunscreen composition in U.S. Pat. No. 5,756,099.

It has been found that the compositions of this invention provide unexpectedly longer control of the appearance of oil and shine on the skin compared to soaps, cleansers, astringents with alcohol, clay, or mud masks. Use of a hydrolyzed protein in cleansing compositions can help reduce oil production of the skin. Reduction of the appearance of oil on the skin by a test composition may be determined as follows. Parallel polarized light digital images are taken of a target skin area, such as the face, immediately after washing the target skin area with a test composition, and then again 4 and 6 hours after washing. Shine assessment is done blindly by an expert grader, who determines if the skin is shiny at each interval.

According to the invention, the appearance of oil on the skin is reduced for at least 2, for example at least 4, preferably at least 6, more preferably at least 8, hours after topical application of a composition of the invention.

The following examples serve to illustrate the compositions and methods of this invention. However, they are not presented in order to limit the scope of the invention in any way.

### EXAMPLE 1

An in vivo study of a composition according to the invention was conducted using two healthy male volunteers, aged 24 and 26 years. The shine on their cheeks was assessed as follows. Composition 3 of Table 1 below was prepared by mixing the ingredients uniformly. The product was applied to the full face of each volunteer after wetting the skin. The product was rubbed on the face for about 10 seconds and followed by a rinse off with warm tap water (˜37° C.) for 15 seconds or until clean. Both subjects noticed a significant reduction of the oil immediately after the wash compared with the baseline, and reported a visible shine control effect for more than 6 hours after the wash.

### EXAMPLE 2

Composition 1 in the Table 1 below (pH=10) was prepared and tested vs. commercially available Clean & Clear® Oil Shine Control (comparative) in a 6 hour facial cleansing study. Fifteen oily subjects with >150 ug/cm2 Sebumeter reading were instructed to wash their faces with Composition 1 on one side of the face, and the comparative cleanser product on the other side of the face. Parallel polarized light digital images were taken immediately after the wash and 4 hours after the wash. Shine assessment was done by an expert, who determined there was significantly less shine for the skin treated with Composition 1 versus the comparative cleanser. The photo also displayed a clear reduction of the oil and shine on the Composition 1-treated side of the faces at 4 and 6 hours after washing. In comparison, at 4 and 6 hours after washing, there was more shine and oil on the comparative sides of the faces.

A comparative composition, Composition A in Table 1, was made by replacing rice protein with aluminum starch octensuccinate and acrylates copolymer (and) magnesium aluminum silicate. When tested using the procedure described above, the shine minimization effect was not observed.

TABLE 1
Composition

| | A | | | | | |
|---|---|---|---|---|---|---|
| Ingredient | (compareative) | 1 | 2 | 3 | 4 | 5 |
| Acrylate polymer | 2.00% | 2.00% | | | 2.00% | |
| Glycerin | | 12.00% | 12.00% | 12.00% | 12.00% | 12.00% |
| Propylene Glycol | | 3.00% | 3.00% | 3.00% | 3.00% | 3.00% |
| Disodium EDTA | | | 0.20% | 0.20% | 0.20% | 0.20% |
| Nipasept | 0.50% | 0.50% | 0.50% | 0.50% | 0.50% | 0.50% |
| Lauric Acid | 6.00% | 6.00% | 6.00% | 6.00% | 6.00% | 6.00% |
| Myristic Acid | 5.00% | 5.00% | 5.00% | 5.00% | 5.00% | 5.00% |
| Stearic Acid | 16.00% | 16.00% | 16.00% | 16.00% | 16.00% | 16.00% |
| G.M. & PEG-100 Stearate | 2.70% | 2.70% | 2.70% | 2.70% | 2.70% | 2.70% |
| Potassium Hydroxide | 5.50% | 5.50% | 5.50% | 5.50% | 5.50% | 5.50% |
| Polyquaternium-39 | 0.80% | 0.80% | 2.50% | | | 2.50% |
| Polyquaternium-7 | | | | 2.50% | 2.50% | |
| Cocamidopropyl PG- | | 0.15% | 0.15% | 0.15% | 0.15% | 0.15% |
| Dimonium | | | | | | |
| Chloride Phosphate | | | | | | |
| Lauryl Glucoside | | 2.00% | 2.00% | 2.00% | 2.00% | 2.00% |
| Lauryl Betaine | | | | | | 3.00% |
| Aluminum Starch | 1.00% | | 0.50% | 0.50% | 0.50% | 0.50% |
| Octenylsuccinate (and) | | | | | | |
| Acrylates Copolymer (and) | | | | | | |
| Magnesium Carbonate | | | | | | |
| Hydrolyzed Rice protein | | 1.00% | 0.50% | 0.50% | 0.50% | 0.50% |
| Magnisium aluminum | 1.0% | | | | | 1.00% |
| silicate | | | | | | |
| Carrageenan | 1.00% | | | | | |
| qs. DI Water to | 100% | 100% | 100.00% | 100.00% | 100.00% | 100.00% |

### EXAMPLE 3

A cleanser according to the invention was prepared by adding 0.5% of hydrolyzed rice protein to commercially available Clean & Clear® Morning Burst cleanser. The resulting cleanser was then tested for its shine control effect on a healthy male (25 years old) volunteer having self perceived oily skin. The subject washed the left side of his face with the composition of the invention and the right side of his face with Clean & Clear® Morning Burst cleanser. Immediately after the wash, the face was free from oil and shine. There was slightly higher residue feeling on the left side of face. The subject reported less oil and shine on his left side of face. This difference was perceived until more than 6 hours after the wash.

### EXAMPLE 4

Composition 7 shown in Table 2 (containing partially hydrolyzed rice protein) was investigated in an in-vivo clinical study. Ten subjects presenting oily and shiny facial skin conditions were recruited based on their self perceived oily skin and sebumeter reading >180 ug/cm2 on their cheek. Each subject washed his face using Composition 7. Visible digital photo images were taken immediately after the wash and at 4 hours and 6 hours after the wash. Expert panel evaluation of the photos demonstrated a visible shine reduction after both 4 hours and 6 hours.

TABLE 2

| | | Composition | B |
|---|---|---|---|
| Ingredient | 6 | 7 | (Comparative) |
| Water | 31.85 | 31.85 | 31.85 |
| Acrylate polymer | 2.00 | 2.00 | 2.00 |
| Glycerin | 12.00 | 12.00 | 12.00 |
| Propylene Glycol | 3.00 | 3.00 | 3.00 |
| Disodium EDTA | 0.20 | 0.20 | 0.20 |
| Methylparaben (and) | 0.50 | 0.50 | 0.50 |
| Ethylparaben (and) | | | |
| Proopylparaben | | | |
| Lauric Acid | 6.00 | 6.00 | 6.00 |
| Myristic Acid | 5.00 | 5.00 | 5.00 |
| Stearic Acid | 16.00 | 16.00 | 16.00 |
| Glyceryl Stearate (and) PEG- | 2.70 | 2.70 | 2.70 |
| 100 Stearate | | | |
| Purified Water | 9.60 | 9.60 | 9.60 |
| Potassium Hydroxide | 5.50 | 5.50 | 5.50 |
| Lauryl Glucoside | 2.00 | 2.00 | 2.00 |
| Polyquaternium-7 | 2.50 | 2.50 | 2.50 |
| Cocamidoproppyl PG- | 0.15 | 0.15 | 0.15 |
| Dimonium Chloride Phosphate | | | |
| Aluminum Starch | 0.50 | 0.50 | 0.50 |
| Octenylsuccinate (and) | | | |
| Acrylates Copolymer (and) | | | |
| Magnesium Carbonate | | | |
| Hydrolyzed Rice Protein | 0.50 | 0.00 | 0.00 |
| Partially Hydrolyzed Rice | 0.00 | 0.50 | 0.00 |
| Protein | | | |
| Rice Extract | 0.00 | 0.00 | 0.50 |
| Total | 100.00 | 100.00 | 100.00 |

### EXAMPLE 5

A toner is made according to the invention as follows using the ingredients shown in Table 3.

Phase A

At room temperature rice protein is dispersed in the water and mixed for a sufficient time until dispersed. Xanthan gum is then added to the dispersion and mixed therein for a sufficient time until the dispersion thickens. Alternatively, xanthan gum can be first dispersed and then the rice protein added. Optionally, 1,3-dibutylene glycol can be added during dispersion formation. The pH of the dispersion is adjusted with an acid to a pH of from about 3 to about 4.5.

Phase B

At room temperature salicylic acid and the alcohol are mixed until dissolved in the alcohol. Optionally, PPG-15 stearyl ether and the fragrances can be added. The pH is adjusted with a base to from about 3 to about 4.

Phases A+B

Phases A and B are mixed and optionally, colorants can be added. The pH can be adjusted by addition of an acid or a base where appropriate to a pH of from about 3.0 to about 4. The target pH at the time of manufacture is about 3.5.

TABLE 3
Toner example

| Ingredient | Wt. % |
|---|---|
| Phase A | |
| Water | 20 |
| Rice protein | 2 |
| Xanthan gum | 0.25 |
| 1,3-dibutylene | 2 |
| glycol | |
| Phase B | |
| Alcohol | 20 |
| Salicylic acid | 2 |
| PPG-15 Stearyl | 2 |
| Ether | |
| Fragrances | q.s. |
| Water q.s. to | 100 |

### EXAMPLE 6

Two cleansing compositions according to the invention, Compositions 6 and 7, and one comparative cleansing composition, Composition B, all described in Table 2, were prepared and tested on 10 oily subjects using the procedure described in Example 1. Compositions 6 and 7 were found to reduce the shine immediately and maintain the shine reduction for more than 4 hours. In contrast, comparative Composition B demonstrated much less shine control effect over time.

### EXAMPLE 7

### Cleansing Bar

Example 7 describes the preparation of a cleansing bar according to the invention for normal-to-oily skin using the ingredients listed in Table 4.

Pre-Mix

Preparation is at room temperature. PEG-14M is dispersed in glycerin, and maintained agitation of the mixture until a creamy homogeneous liquid was formed. Then the ingredients are added in the following sequence into a container with the water under continuous mixing: PEG-14M/glycerin mixture, IPBC solution, potassium sorbate, which was added.

Colloidal oatmeal, rice protein, sodium cocoyl isethionate, magnesium aluminum silicate, titanium dioxide, and encapsulated benzaldehyde are added to a container and mixed for about 2-3 minutes. With the mixer running, the following ingredients are added sequentially: cold premix, lactic acid, sodium lactate, purified water, and benzaldehyde. Cetyl alcohol is added and heated until it attains a melted state. Then the mixture is mixed until the color is uniform, and the mixture attains a lumpy consistency. The pH is adjusted to a desired 3.5 to 4.5 by either lactic acid or sodium lactate. The mixed batch is placed into the feed hopper of a preliminary plodder, and extruded, pressed, and packaged to form cleansing bars.

TABLE 4
Cleansing Bar

| Ingredients | Wt % |
|---|---|
| Pre-mix | |
| PEG-14M | 0.20 |
| purified water, USP | 13.30 |
| glycerin, USP | 1.50 |
| Potassium sorbate, FCC | 0.50 |
| Colloidal oatmeal, USP | 30.00 |
| Rice protein | 8.00 |
| sodium cocoyl isethionate | 31.00 |
| titanium dioxide, USP | 1.00 |
| Magnesium aluminum silicate, NF | 0.50 |
| lactic acid (88%, i.e., 88 wt % lactic acid, | 1.70 |
| 12 wt % water) | |
| Potassium lactate (60%, i.e., 60 wt % potassium | 1.60 |
| lactate, 40 wt % water) | |
| (Quantity of sodium lactate varied to obtain target pH) | |
| cetyl alcohol, NF | 9.00 |
| IPBC liquid | 0.30 |
| benzaldehyde, about 10% encapsulated in polyoxymethylene | 1.00 |
| Isopentylcyclohexanone nopyl acetate, camphylcyclohexanol | 0.40 |
| Total | 100.00 |

### EXAMPLE 8

A study was conducted using the ASTM D1483 method to determine the oil absorption capacity of rice protein versus other commonly used oil absorbers. In this test, a soft paste is formed by the dropwise addition of linseed oil to the gently stirred oil absorbing material in a small glass beaker. The amount of oil required to form the paste is used to calculate an oil absorption value. The results are summarized in Table 5.

TABLE 5

| Material | Ratio of Material:Oil | |
|---|---|---|
| Rice protein | 1:1.1 | g |
| Carregena | 1:2 | g |
| Magnesium aluminum silicate | 1:1.5 | g |
| Aluminum | 1:4 | g |

### EXAMPLE 9

An oil absorbing powder according to the invention is made as follows at 25 to 30° C. and atmospheric pressure using the ingredients shown in Table 6. Premix 1 and premix 2 are prepared separately in different steel containers. Mechanical stirrers mix the mixtures about 10 minutes until they are homogeneous.

Premixes 1 and 2 are added to talc and sprayed via a sprayer such as a 37 SC model Mateer-Burt sprayer. The resultant mixture is further mixed in the same mixer under ambient conditions until homogeneous.

TABLE 6
Oil Absorbing Powder

| | % (wt/wt) |
|---|---|
| Premix 1 | |
| Menthol | 0.15 |
| Eucalyptus oil | 0.25 |
| p-chloro-3,5-m-xylenol | 0.1 |
| Fragrance | 0.18 |
| Premix 2 | |
| Scutellaria baicalensis Extract | 0.005 |
| 1-Carboxy-N,N,N-trimethylmethanaminium hydroxide | 0.5 |
| zinc oxide | 1.0 |
| Rice protein | 10 |
| Preparation of Powder Composition | |
| Talc | 88 |

### EXAMPLE 10

An oil cleansing sheet according to the invention is made of oil absorbing plant fibers such as hemp or synthetic pulp. High-compression roller pressing is carried out during production of the oil cleaning sheets with a paper hardness of at least 0.7 (g/cm²). The surface of the paper is coated with a rice protein powder using the manufacturing process described, e.g., by Japanese Unexamined Utility Model Publication (Kokai) No. 4-45591. Rice protein can enhance the oil absorption and help smooth the oil absorbing sheets hardness and roughness and reduce the irritation associated with such roughness.

### EXAMPLE 11

An oil cleansing sheet according to the invention can be prepared according to Japanese Unexamined Utility Model Publication (Kokai) No. 5-18392. Rice proteins are added to oil cleansing paper to form a smooth side on the surface of the oil cleansing paper. Since these oil cleansing sheets contain particles in the gaps between the fibers of the oil cleansing paper, skin oils moisturize the entirety of the oil cleansing sheet while also filling the gaps between the paper fibers and the particles, providing an effect of rendering the oil cleansing paper even more transparent, i.e., giving the user the satisfactory perception that skin oil has been removed.

While this specification describes the principles of the present invention, it is to be understood that other aspects, advantages and modifications within the scope of the invention will be apparent to those skilled in the art.

What is claimed is:

1. A composition for cleansing skin comprising at least one hydrolyzed protein and a cleansing agent, wherein said composition is a capable of reducing the appearance of oil on the skin for at least 6 hours.

2. A composition according to claim 1, wherein said hydrolyzed protein is selected from rice protein, wheat protein, barley protein, maize protein, millets protein, sorghum protein, oat protein, rye protein, fonio protein, quinoa protein, buckwheat protein, and combinations thereof.

3. A composition according to claim 1, wherein said hydrolyzed protein is present in an amount of from about 0.1 to about 90% by weight of the composition.

4. A composition according to claim 1, wherein said hydrolyzed protein comprises a hydrolyzed protein portion and non-hydrolyzed protein portion.

5. A composition according to claim 4, wherein said hydrolyzed protein portion is present in an amount of from about 0.05% to about 95% by weight of said hydrolyzed protein.

6. A composition according to claim 4, wherein said non-hydrolyzed portion is present in an amount of from about 90% to about 99.9% by weight of said hydrolyzed protein.

7. A composition according to claim 1, wherein said cleansing agent is selected from lauric acid, myristic acid, palmitic acid, steraric acid, their salts, coconut oil with alkali, surfactants, and combinations thereof.

8. A composition according to claim 7, wherein said surfactant is an anionic surfactant selected from sodium lauryl sulfate and sodium lauryl sulfonate.

9. A composition according to claim 7, wherein said surfactant is present in the amount of from about 1.0% to about 90% by weight of the composition.

10. A composition according to claim 1, further comprising an effective amount of a keratolytic agent.

11. A composition according to claim 10, wherein said keratolytic agent is selected from salicylic acid, benzoyl peroxide, resorcinol, colloidal sulphur, selenium disulphide, sulfur, and a combination thereof.

12. A composition according to claim 10, wherein said keratolytic agent is present in an amount of from about 0.01% to about 10% by weight of the composition.

13. A composition according to claim 1, further comprising an effective amount of at least one thickening agent.

14. A composition according to claim 13, wherein said thickening agent is selected from NaCl, NH₄Cl, KCl, Na₂SO₄, clays, silicas, magnesium aluminum silicate, cellulosic polymers, xanthan gum, fatty alcohols, fatty acid esters, fatty acid amides, ethers of polyethylene glycol or sorbitol polyethylene glycol, cocamide of mono- or di-ethanolamide, cocamidopropyl betaine, and combinations thereof.

15. A method for depositing a thin coating of rice protein on skin, comprising topically applying to said skin an effective amount of a delivery system composition comprising a deposition enhancer system and said rice protein.

16. A method according to claim 15, wherein said deposition enhancer system comprises a cationic compound selected from phytantriol, polyquaternium-6, polyquaternium-7, polyquaternium-22, polyquaternium-39, polyquaternium-87, and combinations thereof.

17. A method for reducing the appearance of oil on the skin for at least 6 hours, which comprises topically applying to said skin a composition comprising at least one hydrolyzed protein and a cleansing agent.

18. A method according to claim 17, wherein said hydrolyzed protein is selected from rice protein, wheat protein, barley protein, maize protein, millets protein, sorghum protein, oat protein, rye protein, fonio protein, quinoa protein, buckwheat protein, and combinations thereof.

19. A method according to claim 17, wherein said hydrolyzed protein is present in an amount of from about 0.1 to about 90% by weight of the composition.

20. A method according to claim 17, wherein said hydrolyzed protein comprises a hydrolyzed protein portion and non-hydrolyzed protein portion.

21. A method according to claim 20, wherein said hydrolyzed protein portion is present in an amount of from about 0.05% to about 95% by weight of the hydrolyzed protein.

22. A method according to claim 20, wherein said non-hydrolyzed portion is present in an amount of from about 90% to about 99.9% by weight of the hydrolyzed protein.

23. A method according to claim 17, wherein said cleansing agent is selected from lauric acid, myristic acid, palmitic acid, steraric acid, and their salts, coconut oil with alkali, anionic surfactant, and combinations thereof.

24. A method according to claim 23, wherein said anionic surfactant is selected from lauryl sulfates and lauryl sulfonates.

25. A method according to claim 23, wherein said anionic surfactant is present in the amount of from about 1.0% to about 90% by weight of the composition.

26. A method according to claim 17, further comprising an effective amount of a keratolytic agent.

27. A method according to claim 26, wherein said keratolytic agent is selected from salicylic acid, benzoyl peroxide, resorcinol, colloidal sulfur, selenium disulphide, sulfur, and a combination thereof.

28. A method according to claim 26, wherein said keratolytic agent is present in an amount of from about 0.01% to about 10% by weight of the composition.

29. A method according to claim 17, further comprising an effective amount of at least one thickening agent.

30. A method according to claim 29, wherein said thickening agent is selected from NaCl, NH₄Cl, KCl, Na₂SO₄, clays, silicas, magnesium aluminum silicate, cellulosic polymers, xanthan gum, fatty alcohols, fatty acid esters, fatty acid amides, ethers of polyethylene glycol or sorbitol polyethylene glycol, cocamide of mono- or di-ethanolamide, cocamidopropyl betaine, and combinations thereof.
